(19) United States
(12) Patent Application Publication
(10) Pub. No.: US 2001/0006948 A1
(43) Pub. Date: Jul. 5, 2001

(54) GENE TRANSFER TO INTERVERTEBRAL DISC CELLS

(76) Inventors: JAMES D. KANG, PITTSBURGH, PA (US); CHRISTOPHER H. EVANS, PITTSBURGH, PA (US); KOTARO NISHIDA, PITTSBURGH, PA (US); PAUL D. ROBBINS, PITTSBURGH, PA (US)

Correspondence Address: DIANE R MEYERS, ECKERT SEAMANS CHERIN & MELLOTT, 600 GRANT STREET, 44TH FLOOR, PITTSBURGH, PA, 15219

(21) Appl. No.: 09/199,978

(22) Filed: Nov. 25, 1998

(51) Int. Cl. A61K 48/00

(52) U.S. Cl. 514/44; 435/320.1; 435/325; 435/69.1; 424/193.1; 424/93.1

(57) ABSTRACT

Methods for transferring a gene to an intervertebral disc are disclosed. Such methods find application in the treatment of patients for degenerative disc disorders, by use of a gene encoding a product that imparts a therapeutic and/or prophylactic benefit. The present methods also find application in the establishment of an animal model for the study of degenerative disc disease. A genetically modified intervertebral disc cell is also disclosed.

### FIELD OF THE INVENTION

The present invention is generally directed to methods for introducing a gene into an intervertebral disc cell. The methods generally comprise introducing a nucleic acid sequence encoding a gene of interest into a population of intervertebral disc cells, such that the disc cells subsequently express the gene, thereby producing the product encoded by the gene. The present methods are useful, for example, in the treatment and study of disc disorders.

### BACKGROUND OF THE INVENTION

The intervertebral disc is anatomically and physiologically unique. The disc itself is generally made up of two components, the annulus fibrosus and the nucleus pulposus. The annulus fibrosus is made up of fibrous lamellae, or sheets of collagen fiber. The nucleus pulposus is a deformable mass of watery gel. The annulus surrounds and contains the nucleus pulposus, and helps prevent structural failure. The nucleus pulposus has a very low cell density.

Intervertebral disc degeneration is one of the leading causes of spinal disorders. Disc degeneration and associated spinal disorders are a leading source of morbidity resulting in substantial pain and suffering and increased health care costs.

Intervertebral disc changes accompany the aging process, and are therefore inevitable. These changes include, for example, the loss of water from the nucleus pulposus, disc splits and clefts, disc height loss, disc protrusion and disc herniation. Minor injuries and traumas over time also contribute to disc degeneration, serving to alter the disc's structural integrity. More severe injuries and traumas can also contribute to the acceleration of disc degeneration.

Although the etiology and pathophysiology of intervertebral disc degeneration are largely unknown, some evidence indicates that disc degeneration can originate in the nucleus pulposus, with progressive decrease in proteoglycan content leading to dehydration of the nucleus pulposus. Because the swelling pressure resulting from the high concentration of proteoglycans in the nucleus pulposus helps to maintain disc height and contributes to the load bearing ability of the disc, loss of proteoglycans may directly affect the biomechanical function of intervertebral discs as well as alter loading of the facet joints and other structures, leading to degenerative changes.

Disc tissues have a very limited ability to regenerate. Therefore, once the degeneration process has started, it is difficult to stop or reverse the process with any currently available techniques. Although the mechanisms are not well known, some cases of disc degeneration progress faster than the aging process, for example, secondary degeneration in discs adjacent to long fusion, or in discs following annulus injury.

Few approaches are available clinically for the treatment or prevention of disc degeneration, and the disc appears to have a limited ability to repair itself. The limited available technology for the treatment of intervertebral disc disorders is generally high invasive, and usually requires surgery in which all or portions of the affected intervertebral disc or discs are removed. Examples of this surgery include nucleotomy and discectomy; both of these procedures, however, compromise the structural integrity of the disc, necessitating introduction of materials to restore mechanical stability to the altered structure, such as autologous bone graphs and/or spinal instrumentation such as pedicle plates or screw systems. Non-invasive treatments, such as bed rest, medication, spinal manipulation, traction and the like tend to relieve the symptoms associated with disc degeneration rather than treating the disc itself.

There have been some reported attempts to treat spinal disorders. For example, Wehling and colleagues have reported transfer of genes to chondrocytic cells of the lumbar spine. More specifically, chondrocytic cells were removed from intervertebral end plates, cultured in vitro, and genetically modified by addition of cDNA encoding for various genes. Wehling does not appear to teach direct in-vivo or ex-vivo gene transfer into disc cells.

Reinecke and colleagues have similarly demonstrated the transfer of therapeutic genes to human chondrocytic-like cells derived from prolapsed lumbar disc. Again, there is no teaching or introduction of exogenous genes into the intervertebral disc cells themselves.

Other approaches to the treatment of acute disc injury or other disc disease include direct injection of growth factor proteins or appropriate carriers combined with proteins. Growth factors are polypeptide-signaling molecules that can stimulate cells to grow or proliferate, among other things. Growth factors have been tested in animal models and are reported to enhance tissue repair. For example, Boden and colleagues have reported the use of osteoinductive growth factors for lumbar spinal fusion. While increased fusion rates in a rabbit lumbar fusion model using bone morphogenetic proteins (BMP) were shown, no gene therapy is taught or suggested. Spine, 20(24):2626-31 (1995); Spine, 20(24):2633-44 (1995) and Spine, 23(3):291-96 (1998) (discussion 7). Direct injection of growth factor, however, has decided shortcomings in the treatment of disc degeneration, as growth factors have a relatively short half life; in addition, there are problems associated with diffusion in the tissue which appear to limit the usefulness of direct injection of growth factors for treatment of ailments of the intervertebral disc.

Chronic types of diseases such as disc degeneration require a sustained delivery of exogenous growth factors, or other genes, to the disc. Such methods have not been previously reported. There remains a need, therefore, for methods of prophylactically and therapeutically treating degenerative disc disease by sustained delivery of exogenous genes. There also remains a need for animal models useful in the study of degenerative disc disorders.

### SUMMARY OF THE INVENTION

The present invention has met the above-described needs by providing methods for introducing a gene or genes of interest to the intervertebral disc cells of a patient. The methods generally involve introducing a nucleic acid sequence encoding a gene of interest into a population of intervertebral disc cells (IDCs), such that the gene will be expressed by the IDCs. The genes delivered to the IDCs can encode proteins, peptides, polypeptides or types of RNA useful in a number of applications. For example, the gene of interest can encode a product useful in the treatment of a patient for a degenerative disc disease. Alternatively, the gene of interest can encode a product which causes pathology of the disc, which would be useful in creating an animal model for study of degenerative disc disorders. Genetically modified IDCs are also within the scope of the present invention.

It is, therefore, an object of the present invention to provide a method for introducing a gene of interest into an intervertebral disc cell of a patient.

It is the further object of the invention to provide such methods wherein an animal model for study of degenerative disc disease is produced.

Another object of the invention is to provide such methods useful in the therapeutic and/or prophylactic treatment of degenerative disc disease.

Another object of the invention is to provide intervertebral disc cells that have been genetically modified by the introduction of an exogenous gene.

Another object of the invention is to provide methods for regeneration of intervertebral disc tissue.

Yet another object of the invention is to provide methods for treating patients having degenerative disc disease, wherein such methods of treatment are generally non-invasive.

These and other objects of the present invention will be apparent to those skilled in the art based upon the following disclosure and appended claims.

### BRIEF DESCRIPTION OF THE DRAWINGS

The file of this patent contains at least one color photograph. Copies of this patent with color photographs will be provided by the Patent and Trademark Office upon request and payment of the necessary fee.

FIG. 1 shows the adenovirus mediated β-galactosidase (LacZ) expression in nucleus pulposus cells in vitro after three weeks culture, determined according to the methods of Example 1.

FIG. 2 shows the adenovirus mediated β-galactosidase (LacZ) expression in nucleus pulposus cells in vivo after three weeks (FIG. 2a) and eight weeks (FIG. 2b) post-injection, determined according to the methods of Example 1.

FIG. 3 shows the bioassay results for active TGF-β1 production, determined according to the methods of Example 2.

FIG. 4 shows the bioassay results for proteoglycan synthesis, determined according to the methods of Example 2.

### DETAILED DESCRIPTION OF THE INVENTION

The present invention is generally directed to a method for transferring a gene of interest to an intervertebral disc cell of a patient comprising introducing a nucleic acid sequence encoding a gene of interest to an intervertebral disc cell, such that the gene will be expressed by the intervertebral disc cell.

It will be appreciated that genes encode proteins, peptides, polypeptides, and therapeutic species of RNA including but not limited to antisense RNA, decoy RNA, and ribozymes, all of which are collectively referred to herein as “products”. A “nucleic acid sequence of interest”, “gene of interest” or “product of interest” refers to nucleic acid sequences, genes, or the products they encode that are introduced into an intervertebral disc cell according to any of the methods of the present invention. A gene of interest can be selected based on the function of the product that it encodes. For example, when the present methods are utilized in the therapeutic or prophylactic treatment of a host, the gene of interest would encode products, or biologically active fragments or derivatives thereof, that have therapeutic and/or prophylactic properties in the treatment of degenerative disc diseases. Similarly, genes that block pain could also be used, as could anti-apoptotic genes. When the present methods are used in creating an animal model, the gene of interest would encode products, or biologically active fragment or derivatives thereof, that have a pathologic effect on the host, contributing to one or more of the deleterious effects of degenerative disc diseases. Thus, the “gene of interest” varies depending on the particular application desired by the user.

As used herein, the terms “patient” and “host” include vertebrates, including, but not limited to, human beings.

The term “biologically active” refers to the ability of a product to bring about a known or expected biological function. As will be appreciated by those skilled in the art, a fragment or derivative of a nucleic acid sequence, gene or a product of interest can still function in the same biological and/or physiological manner as the entire wild type sequence, gene or product. “Biologically active” fragments or derivatives of nucleic acid sequences, genes and products of interest are therefore also within the scope of the present invention. For example, a form of a nucleic acid sequence can have variations as compared with the wild type sequence, but the sequence will still encode a product that retains its wild type function despite these variations. Similarly, derivatives of genes and products of interest used in the present invention will have the same biological effect on the host as the non-derivatized forms. Examples of such derivatives include, but are not limited to, dimerized or oligomerized forms of the genes or products, and genes or products modified by the addition of an immunoglobulin molecule or a part of an immunoglobulin molecule (IgG). One skilled in the art can test for the biological activity of a derivative or fragment of sequences, genes or products of interest by various methods known to those skilled in the art. For example, the biological activity of a wild type form can be compared with a fragment or derivative and, if the same or similar result is obtained, the fragment or derivative is also biologically active.

As stated above, any of numerous genes can be used according to the present invention, depending on the needs of the user. In addition, it is possible to transfer more than one gene of interest to an IDC of a patient according to the present methods. This can be accomplished, for example, by introducing more than one nucleic acid sequence, each of which encodes a different gene of interest. In this manner one or more products will be produced by the IDC. Products can be selected, therefore, to achieve additive, synergistic, or complementary effects.

Numerous methods for introducing the nucleic acid sequence into the host can be used. For example, both viral and non-viral methods can be employed. These methods, described below, are referred to collectively herein as “delivery means” or “delivery systems”. Typically, regardless of the delivery system used, a suitable medium or carrier containing the delivery system will be used to introduce the nucleic acid sequence into the IDCs. A suitable carrier is one which is physiologically compatible with the delivery means being used and which will not have a deleterious effect on the patient and can be a solution, such as saline or Gey's Balanced Salt Solution (GBSS), a gel, a colloid, or other carriers known in the art.

Viral methods involve utilization of a viral vector, engineered or constructed to contain a nucleic acid sequence or sequences of interest. Numerous viral vectors are known in the art, and are suitable for use with the present methods. This includes, for example, retroviral vectors, adeno-associated viral vectors, adenoviral vectors, and herpes virus vectors, such as herpes simplex virus vectors. Viral vectors for use in introducing genes to a cell are generally described in Robbins, et al., Tibtech, 16: (1998); and P. D. Robbins and S. C. Ghivizzani, “Viral Vectors for Gene Therapy”, Pharmacol Ther. 80:35-47 (1998).

Retroviral vectors are constructed using genes from a retrovirus. Examples of suitable retroviral vectors include the MFG retroviral vector and the pLJ retroviral vector. An MFG vector is a simplified Moloney murine leukemia virus vector (MoMLV) in which the DNA sequences encoding the pol and env proteins have been deleted so as to render it replication defective. An MFG vector can be prepared that contains a nucleic acid sequence of interest. An MFG vector having two nucleic acid sequences (DFG), three nucleic acid sequences (TFG) or even more nucleic acid sequences of interest can be included in the MoMLV. Thus, DFG and TFG are forms of MFG having multiple nucleic acid sequences. For ease of reference, the term MFG, as used herein, includes any singular or multi-gene form of the vector. The pLJ retroviral vector is also a form of MoMLV and is more fully described by Korman et al., Proc. Nat'l Acad. Sci., 84:2150-2154 (1987), which description is hereby incorporated by reference. Another preferred retrovirus for use in the present invention is the lentiviral vector which is based on, for example, the human immunodeficiency virus (HIV), the simian immunodeficiency virus (SIV), or equine infectious-anaemia virus (EIAV); the lentivirus has the capability of infecting non-dividing cells.

Adenovirus and adeno-associated virus vectors can also be used. Adenoviral vectors can be converted for use in gene transfer by deleting the E1 gene; these vectors can infect a wide variety of cell types and can be grown to high titers. Adeno-associated virus is a member of the parvovirus family and can be used to introduce a gene of interest by inserting the gene between the two inverted terminal repeats required for viral replication.

The herpes virus vector can be prepared from, for example, the herpes simplex type I or herpes simplex type II virus. These large, linear DNA viruses (approximately 150 kb) are able to infect cells lytically and to establish latency in specific cell types. Replication defective herpes virus should be used in making the vector.

Any of the above viral vectors, or others known in the art, can be prepared for use in the methods of the present invention. It will be understood by those skilled in the art that the vector construct contains, in addition to one or more nucleic acid sequences, a promoter which functions in intervertebral disc cells. The selection of an appropriate promoter will vary depending on the viral vector used. A preferred vector for use in the methods of the present invention is the adenoviral vector. The cytomegalovirus (CMV) promoter is a suitable, and preferred, promoter for use in conjunction with the adenoviral vector. The long terminal repeat (LTR) promoter is preferred for use with retroviral vectors.

Introduction of one or more nucleic acid sequences can also be effected by non-viral means. Any non-viral means known in the art can be used. Examples include the use of a liposome, calcium phosphate, electroporation, DEAE-dextran, and direct injection of naked DNA or RNA. It will be appreciated that these non-viral means for introducing the gene of interest into the intervertebral disc cells are non-infectious delivery systems. An advantage of the use of a non-infectious delivery system is the elimination of insertional mutagenesis or other virally induced disease. It will be appreciated by those skilled in the art that the vectors employing a liposome are not limited by cell division for efficient transfection of intervertebral disc cells.

The liposome can be a material selected from the group consisting of DC-chol, SF-chol and numerous others known to those skilled in the art. The term “DC-chol” means a cationic liposome containing cationic cholesterol derivatives. The DC-chol molecule includes a tertiary amino group, a medium length spacer arm (2 atoms) and a carbamoyl linker bond as described in Biochem. Biophys. Res. Commun., 179:280-285 (1991). “SF-chol” is also a type of cationic liposome.

Direct injection of naked nucleic acid sequences can also be used, and involves injection of the nucleic acid contained within a suitable carrier. Direct injection is a preferred non-viral delivery means.

The present methods include transferring genes of interest to IDCs by either in vivo or ex vivo means. In vivo methodologies utilize the direct introduction of viral or non-viral delivery means directly to the intervertebral disc cells. Ex vivo methodologies involve the introduction of delivery means to intervertebral disc cells that have been removed from a patient and cultured in vitro; the cultured IDCs are then returned to the disc of a patient. As used herein the term “intervertebral disc cells” refers collectively to annulus cells and nucleus pulposus cells. The present methods for transferring a gene of interest to the intervertebral disc cells, therefore, contemplates introduction to either annulus cells, nucleus pulposus cells, or both. Nucleus pulposus cells are preferred target cells as they appear to take up and express genes better. In the ex vivo methods, other cells could also be removed from a patient and returned to the disc, including but not limited to stromal cells from bone marrow and other progenitor cells.

As an example, but not a limitation, of an in vivo embodiment of the present invention, a needle can be used to inject the delivery means directly into the disc of the patient such that contact between the delivery means and the IDCs is effected. For example, viral vectors contained in a suitable medium can be directly injected into the nucleus pulposus of the patient. Similarly, naked DNA or RNA sequences contained in a suitable medium can be directly injected into the disc of the patient.

Alternatively, an ex vivo methodology can be employed. Generally, this involves removal of IDCs from the patient and subsequent culture of these IDCs in vitro. Stromal cells or other progenitor cells could also be used. Cultured cells are then contacted with the nucleic acid sequence. For example, a viral vector contained within a suitable medium can be added to the culture; similarly, naked DNA or other nucleic acid sequences contained in a suitable medium can be added to the culture. An incubation period coupled with gentle agitation during a portion of the incubation period is generally sufficient for transfer of the nucleic acid sequence of interest into the IDCs. As an example of a preferred embodiment, adenoviral vectors containing a nucleic acid sequence of interest in GBSS medium is introduced to a culture of nucleus pulposus cells. The culture is incubated at a temperature of about 37° C. for about one hour, with gentle agitation of the viral supernatant during the middle third of the incubation period.

It will be appreciated that in both the in vivo and ex vivo methodologies the nucleic acid sequence, contained either in the viral or non-viral delivery system, need not be directly injected into the IDCs. Rather, the IDCs are capable of taking up the delivery means such that the genes encoded by the nucleic acid sequence contained in the delivery means will be expressed. For example, when using a viral delivery means, the virus is designed to bind to cells and become internalized by them; similar results are seen when using non-viral delivery means. Thus, it will be appreciated that to effect transfer of a nucleic acid sequence of interest according to the method of the present invention, one must just contact the delivery means containing the nucleic acid sequence with the intervertebral disc cells.

In vivo methodologies are preferred for use in all of the methods of the present invention. It will be appreciated that direct injection of the delivery means into the disc cells of the patient bypasses the requirement of removing, in vitro culturing, transfection or transduction, selecting, and transplanting the transfected or transduced IDCs back into the patient. As illustrated more fully in the examples below, the in vivo methodologies result in the transfer and expression of genes of interest to a significant number of IDCs.

The methods of the present invention have numerous applications, and can be used, for example, in the treatment of a patient. The present invention is therefore further directed to a method for treating degenerative disc disease in a patient comprising introducing an effective amount of a nucleic acid sequence encoding a gene of interest into a population of intervertebral disc cells, such that subsequent expression of the gene by at least one of the intervertebral disc cells in the patient reduces at lease one pathology of degenerative disc disease in the patient.

The term “degenerative disc disease” is used throughout the specification to refer to intervertebral disc disorders that are chronic in nature. This includes, for example, the natural disc degeneration that accompanies the aging process. This also includes disorders that are the result of spinal injury, spinal fusion, and the like. “Disc degeneration” in general is defined as the loss of water from within the disc, with resulting distortion of the nucleus pulposus. Such changes can precipitate the onset of Schmorl's nodes, which are herniations of nuclear material into vertebral bodies. Distortions in the nucleus pulposus can also contribute to disc trauma brought about by strain or flexion. Continued degeneration can contribute to the formation of structural defects and subsequent osteoarthritic changes of the spine.

The treatment effected by this method reduces at least one pathology of degenerative disc disease in a patient. The products of interest are selected based on their ability to effect a biological influence within the disc, thereby producing a desired effect. Such beneficial effects include, but are not limited to, maintenance of proteoglycan content, preservation of disc height, restoration and/or preservation of disc/spine function and structure, and preservation of matrix integrity and content. Clinically, the terms “treatment” and “treating” refer to both therapeutic and prophylactic methods of treatment. While it will be appreciated that there is some overlap between “therapeutic” and “prophylactic” treatments, and the uses of those terms, a therapeutic treatment generally involves the delivery of a gene or genes encoding products that result in the improved biological and mechanical function of the disc, such as by stimulating matrix growth. Gene products for the prophylactic use or prevention of disc disease would impart to the disc cells the ability to manufacture products of interest at an increased level, inhibit matrix loss, and preserve the integrity of the disc tissue at all levels, thereby reducing the likelihood that biological and mechanical disc functional problems would arise, or worsen; thus the present invention provides an effective disc disorder prevention technique. Still other treatment genes include those which block pain and those which protect cells from apoptosis.

“Matrix” refers to extracellular components of the disc, and includes, for example, water, proteoglycans, and collagen. One way in which matrix production is stimulated, therefore, is by the production of proteoglycans. Proteoglycans are one component of the matrix whose stimulation can be regulated. As noted above, the swelling pressure resulting from the high concentration of proteoglycans in the nucleus pulposus helps to maintain disc height and contributes to the load bearing ability of the disc. Loss of proteoglycans, therefore, may directly affect the biomechanical function of the intervertebral discs, as well as alter the loading of the facet joints and other structures. This leads to degenerative changes. Introduction of a gene or genes encoding products that stimulate production of proteoglycan or other matrix components reduces a pathology associated with degenerative disc disease.

The gene or genes of interest utilized in the treatment methods, therefore, vary depending on the type of treatment desired, and the illness and severity of the illness in the patient. For example, genes that encode products which stimulate matrix production can be utilized. Similarly, genes that will inhibit breakdown, or further breakdown, of the disc itself can be used. The present treatment methods therefore contemplate use of gene products that preserve the function of disc cells as well as gene products that prevent the dysfunction of disc cells. Use of more than one gene of either type, as well as combinations of such gene types, is also within the scope of the present invention. Any gene capable of maintenance and expression, and encoding a product of interest having a therapeutic and/or prophylactic effect in the treatment of the pathologies associated with degenerative disc disease, can be used in the present methods of treating a patient. Particularly preferred are these genes that enhance matrix deposition or prevent matrix loss. Genes that encode pain blockers and anti-apoptotic genes are also within the scope of the invention.

Growth factors are an example of products providing a therapeutic benefit by helping to stimulate production of matrix and/or components of the matrix. Growth factors are extracellular polypeptide signaling molecules that can stimulate cells to grow or proliferate, among other actions. Growth factors include, but are not limited to: transforming growth factor β (TGF-β), including the subtypes TGF-β1, TGF-β2 and TGF-β3; fibroblast growth factor (FGF), including the subtypes aFGF and bFGF; and insulin-like growth factor (IGF), including the subtypes IGF-1 and IGF-2. Of the transforming growth factors, TGF-β1 is preferred in the treatment methods of the present invention. TGF-β1 is a particularly multi-functional polypeptide growth factor. While specific receptors for this protein have been found on almost all mammalian cells, the effect of the molecule varies depending on the cell type, growth conditions, state of cell differentiation, and presence of other growth factors. TGF-β1 is an important mediator of the formation of extra cellular matrix, and, therefore finds particular application in the therapeutic and prophylactic methods of the present invention. More specifically, as is illustrated in the examples below, TGF-β1 has been found to stimulate proteoglycan synthesis in IDCs. Similarly, IGFs are known to maintain the synthesis of the extracellular matrix, and promote production of this matrix. Bone morphogenetic proteins (BMP) are an additional type of growth factor. There are at least twelve BMPs; the BMPs are members of the TGF-β superfamily. BMPs are known to induce the formation of both bone and cartilage, and therefore may contribute to the ability of the disc matrix to regenerate. BMP 7 is also known as osteogenic protein-1 (OP-1); BMP-7 and BMP-2 have been shown to be particularly promising in the therapeutic treatment of articulate cartilage. Other growth factors include platelet-derived growth factor (PDGF) and epidermal growth factor (EGF). Isoforms of all of the above growth factors can also be used.

Growth hormone is also a product of interest which can be utilized in the treatment methods of the present invention. Growth hormone is believed to act by inducing local synthesis of IGF-1, although the inventors do not wish to be bound by this mechanism.

In addition to utilization of genes that stimulate production of matrix or various matrix components, the treatment methods of the present invention include use of genes that inhibit breakdown, or further breakdown, of the biological and mechanical functions of discs. Examples of such genes include interleukin-1 receptor antagonist protein (IL-1Ra), soluble interleukin-1 receptors (sIL-1R), soluble TNF-α receptors (sTNF-αR), and proteinase inhibitors.

IL-1Ra is a cytokine known to suppress the inflammatory and destructive responses caused by interleukin-1 (IL-1). It is believed that the IL-1Ra binds to interleukin-1 (IL-1) receptors. This prevents binding of the IL-1 to the receptors and inhibits the deleterious effects caused in discs when IL-1 binds to the receptors. The inventors do not wish to be bound by this mechanism, however.

sIL-1R also interferes with the action of IL-1, but by binding to the IL-1 itself. This prevents the IL-1 from binding to the native, cell surface of IL-1 receptors. Any of the sIL-1 receptors can be used, including, but not limited to, Type I and Type II receptors; sIL-1R type II receptors are preferred because they do not bind to IL-1Ra, while Type I receptors do. Type I sIL-1R is an 80 Kd glycoprotein that is present on T-lymphocytes, fibroblasts and chondrocytes. Type II sIL-1R is 67 Kd in size and is found predominately on macrophages and pre-B-cells.

Soluble tumor necrosis factor-α receptor (sTNF-αR) binds TNF-α and prevents it from having a damaging effect on the disc matrix of the patient. TNF-α is a cytokine which is known to contribute to the pathological effects of degenerative disc disease. The sTNF-αR used in the methods of the present invention can be of any type, including Type I and Type II. The Type I sTNF-α receptor is a 55 Kd glycoprotein and Type II sTNF-R is 75 Kd in size. Both receptors are widely distributed on various cell types.

Various proteinase inhibitors are also within the scope of the present invention. Proteinase inhibitors are substances that prevent the enzymatic breakdown of proteins. Proteinase inhibitors include but are not limited to metalloproteinase inhibitors, all of which are within the scope of the present invention. Preferred proteinase inhibitors are the tissue inhibitors of metalloproteinase (TIMP), of which there are at least four types, TIMP-1, TIMP-2, TIMP-3 and TIMP-4. Other preferred proteinase inhibitors include the plasminogen activator inhibitors (PAIs) and serpins.

Other therapeutic/prophylactic genes include therapeutic cytokines and their derivatives such as IL-10, and IL-4.

Genes that encode pain blocking proteins can also be used. Expression of such genes in a patient would have the effect of reducing and/or eliminating pain in a patient. Any genes expressing such proteins could be used, including but not limited to enkephalines, endorphins, and mutant neuronal receptors. Also within the scope of the invention is the use of anti-apoptotic genes, which would inhibit intervertebral disc cell death. Examples of such genes include but are not limited to Bc1-2, Bc1-X1, and dominant negative (“dn”) genes such as dnFas, dnFlip, dnFlice, and dnFadd.

Any of the delivery means described above can be used to introduce one or more nucleic acid sequences of interest to the intervertebral disc cells of a patient. While the delivery means can be, for example, injected into IDCs, it is also possible to coat a support device with the delivery means and implant or otherwise introduce the support device to the disc of a patient. The delivery means could also be otherwise contained on or in the support device such that it will be released into the disc. For example, a resorbable scaffold or carrier could be coated with delivery means and implanted in the annulus of a disc. The choice of support device material is based on its biocompatability, biodegradability, mechanical properties and interface properties. One skilled in the art can determine the appropriate material based, for example, on the biodegradability and biomechanical properties of the support structure and the condition being treated. Any suitable support device capable of introducing the delivery means to the disc can be used according to the methods of the present invention. Preferably, the support device is capable of being resorbed into the body. For example, support device scaffolds or carriers can be prepared using one or more of collagen, glycosaminoglycans, polylactic acid polymers, such as open cell polylactic acid polymers (OPLA), biodegradable and chemically defined calcium sulfate, tricalciumphosphate, hydroxyapatite, biphasic tricalciumphosphate/hydroxyapatite ceramic, polylactic acids, polylactate, polyanhydrides and chitosan. An absorbable collagen sponge is commercially available from Integra Life-Sciences Corporation under the name Helistat® Absorbable Collagen Hemostatic Agent. Pure proteins or extracellular matrix components can also be used in support devices. Other support devices could also be used, depending on the desire of the user. A metal cage fusion device containing the delivery means can also be implanted in the disc. When using a support device, gene therapy is effected by contacting the annulus and/or nucleus pulposus cells with the delivery means, while mechanical protection of the disc is concurrently achieved. In a related embodiment, the use of a support device in the annulus can be used in conjunction with introduction of the nucleic acid to the nucleus pulposus as taught herein.

An effective amount of nucleic acid, contained in a delivery means, should be employed. The term “effective amount” is used herein to refer to the amount of nucleic acid needed to effect expression of the gene of interest in the disc cells and bring about the desired result in the patient. The effective amount of nucleic acid contacted with the intervertebral disc cells can be determined by one skilled in the art based upon numerous variables, including the reason for the administration. Generally, when employing the present invention in a method for treating a patient, an amount of nucleic acid sequence should be delivered to the patient to effect subsequent expression of the gene of interest at such a level so as to observe at least some therapeutic and/or prophylactic benefit—that is, a benefit that will reduce or contribute to the reduction of at least one pathology of degenerative disc disease. The amount of nucleic acid to introduce will vary from patient to patient depending on such factors as the size of the patient, the type of degenerative disc disease, the severity of the disease, the gene or genes being used, the delivery means being used, and whether the method is being used therapeutically or prophylactically. A therapeutic and/or prophylactic response is typically seen based on delivery of nucleic acid sequences in a manner sufficient to give gene expression in the high pico- to low nanogram range. One skilled in the art can determine the amount of delivery means containing the nucleic acid sequence or sequences to administer to a patient to achieve these levels of expression based on the factors listed above. When the delivery means is a viral vector, introduction in a titer of about 10⁵ transducing units/ml is typically sufficient, but high titers (equal to or greater than about 10⁷ transducing units/ml) are preferred.

As an embodiment of a preferred method for treating degenerative disc disease according to the present invention, an adenoviral vector is prepared which utilizes the CMV promoter and contains the nucleic acid sequence for TGFβ1 (Ad/CMV-TGF-β1). Approximately 10⁶ PFU of the vector contained in saline is injected through a 26-gauge hypodermic needle into the nucleus pulposus of a patient. In this manner, in vivo transduction of intervertebral disc cells is effected; the IDCs subsequently express TGF-β1 and an increase in the synthesis of proteoglycan is observed. It will be understood by those skilled in the art that this example represents merely one preferred embodiment of the present invention. Numerous delivery means and genes can similarly be used, in either in vivo or ex vivo methodologies within the scope of the present invention, and could be introduced both to annulus cells or nucleus pulposus cells or combinations thereof.

Any type of nucleic acid sequence encoding a gene of interest can be used according to the methods of the present invention. Typically, the nucleic acid sequence will be a cDNA, although RNA, oligonucleotides, and anti-sense nucleic acid molecules such as antisense growth factors, cytokines and matrix molecules can also be used. The term “nucleic acid sequence” is therefore intended to encompass sequences of all of these different forms of nucleic acids, with the DNA form being preferred for the methods of the present invention.

The methods of the present invention also have application in the establishment of an animal model to study degenerative disc disease pathologies and models of degeneration. In addition, animal models can be utilized to evaluate expression levels achieved using different delivery means. Accordingly, the present invention is further directed to a method for preparing an animal model for degenerative disc disease comprising introducing an effective amount of a nucleic acid sequence encoding a gene of interest into at least one intervertebral disc cell of the animal, such that subsequent expression of the gene by at least one of the intervertebral disc cells contributes to at least one pathology of degenerative disc disease. Examples of pathologies which can be studied according to the animal models of the present invention include proteoglycan breakdown, collagen breakdown, inhibition of matrix synthesis, and disc cell apoptosis and necrosis.

The animal model methods of the present invention can utilize any of the delivery means described above, which delivery means can again be introduced by either ex vivo or in vivo methodologies. The gene of interest for use in the animal model will be those which cause and/or contribute to the pathologies associated with degenerative disc disease. Any gene that contributes to one or more of the symptoms of degenerative disc disorders can therefore be used in the animal model. As with the therapeutic/prophylactic treatment methodology, more than one gene can be introduced. Genes suitable for use in the animal model methods of the present invention include any genes which cause such a symptom of a degenerative disc disease, including, but not limited to: various forms of interleukin which cause or contribute to degenerative disc disease; tumor necrosis factor (TNF); NO synthase including any of its isoforms; proteinases, including, but not limited to, aggrecanase; and matrix metalloproteinases such as collagenase, gelatinase and stromelysin. These gene products are useful for animal models of degenerative disc disease as they cause or contribute to one or more of the symptoms of the disease. For example, metalloproteinases degrade matrix components such as proteoglycans and collagen. IL-1 and TNF inhibit matrix synthesis and stimulate matrix breakdown. NO synthases may be involved in matrix disturbances and cell death. The gene of interest can also be a marker gene such as β-galactosidase (LacZ) which can be used to study the efficacy of various delivery means, the extent to which a gene introduced to a disc cell will spread, and the like. The present animal models are suitable for use in the testing of different therapeutic agents.

Any of the viral or non-viral delivery means described in conjunction with the therapeutic/prophylactic methods can be used to effect delivery of the nucleic acid sequence or sequences of interest in the animal model. When employing the methods of the present invention in an animal model for the study of degenerative disc disease, an effective amount of nucleic acid refers to that amount of nucleic acid that should be delivered to effect one or more of the symptoms of degenerative disc disease. It will be appreciated that introduction of an effective amount of the deleterious genes listed above, or any others known to those skilled in the art, will result in conditions seen in animals suffering from degenerative disc disorders. The afflicted animal can then be used to study potential methods for therapeutically treating such disorders experienced by humans. Correlation between these animal models and human would be apparent, as all of the animals utilized in the model would, like humans, be in the same phytogenic class of vertebrates. Thus, the animal model of the present invention provides a correlatable means of studying connective tissue disorders.

Finally, the present invention is directed to a genetically modified intervertebral disc cell that expresses an exogenous gene of interest. Such a genetically modified IDC can be prepared by contacting one or more IDCs with one or more nucleic acid sequences encoding the gene or genes of interest. The nucleic acid sequence can be contained in any delivery means, as discussed above.

The methods of the present invention provide advantages over the currently available technology in many respects. The introduction of nucleic acid sequences of interest directly into the intervertebral disc cells allows gene products to be expressed by the cells for sustained periods of time. Therapies which depend on injection or other means of introducing the actual gene product, such as injection of traditional proteinaceous or non-proteinaceous drugs, are only a transient; in contrast, the present inventions provide sustained periods of expression of the gene product for periods of several months, thereby obviating the need for repeated applications of the therapy. Another novel feature of the treatment methods of the present invention relates to the unusual nature of the disc; because the disc is a highly avascular, encapsulated system having limited access to cells of the immune system, treatment by direct gene therapy is ideal. The technique is also highly versatile, in that different genes can be integrated within the delivery means to achieve a variety of useful prescribed mechano-biological effects. In the treatment of intervertebral disc disorders, the present methods provide a therapy that can be accomplished on an out-patient basis and by a minimally invasive surgical technique, such as percutaneous injection. In the prophylactic treatment of degenerative disc disease, the present methods impart to the disc cells the ability to manufacture therapeutic proteins and other gene products at an increased level, reducing the likelihood that disc biological and mechanical functional problems would arise. Present “techniques” or “recommendations” for the prevention of intervertebral disc degeneration include such things as losing weight, quitting smoking, not lifting heavy weights, using more ergonomically designed chairs, and the like. Finally, the present methods provide a useful and reliable animal model for the study of degenerative disc disorders by delivery of genes encoding the relevant mediators of pathology; no such animal model has been previously reported.

### EXAMPLES

The following examples are intended to illustrate the present invention and should not be construed as limiting the invention in any way.

All of the experimental protocols were approved by the Institutional Animal Care and Use Committee and the Institutional Biosafety Committee at the University of Pittsburgh.

### Example 1

Skeletally mature New Zealand white female rabbits, weighing 4.0-5.0 lbs., were used in this study. After the animals were sacrificed, portions of the nucleus pulposus were carefully isolated from lumbar intervertebral discs. The disc cells were then harvested from nucleus pulposus using 0.01% collagenase treatment for 30 minutes followed by centrifugation at 1500 rpm. The primary cultures were sustained for 4 weeks in Dulbecco's Modified Eagles Medium (DMEM) containing 10% fetal bovine serum (FBS) after which the cells were subcultured in 24 well plates. The culture medium was changed regularly every week.

At confluence, 50 μl of GBSS containing 5×10⁶ PFU of adenovirus (Ad/CMV-LacZ;MOI=200) was added to each culture. The adenovirus used was one in which the E1 and E3 regions of the adenovirus genome were deleted; the LacZ gene was cloned into the E1 region of the adenovirus under the control of the CMV early promoter. The cultures were incubated at 37° C. for one hour with gentle agitation of the viral supernatant during the middle third of the incubation period. For the control group, 50 μl of GBSS without adenovirus was added to the culture. After one hour, 1 ml of DMEM medium was added to each well, and the cells were further incubated at 37° C. The transfected gene expression was examined at one and three weeks, respectively, by using 5-bromo-4-chloro-3-indolyl-β-galactosidase (X-gal) staining technique.

Under general anesthesia, the anterior aspect of intervertebral discs (L2-3, L3-4, L4-5) of the rabbits were exposed using a retroperitoneal approach. 20 μl of GBSS containing the Ad/CMV-LacZ virus particles (6x10⁶ PFU) were injected into the nucleus pulposus through a 26-gauge needle. The control group was injected with 20 μl of normal GBSS. The animals were sacrificed at one, three and eight weeks, respectively, and the intervertebral discs were harvested for histological evaluation for X-gal staining supplemented with eosin staining.

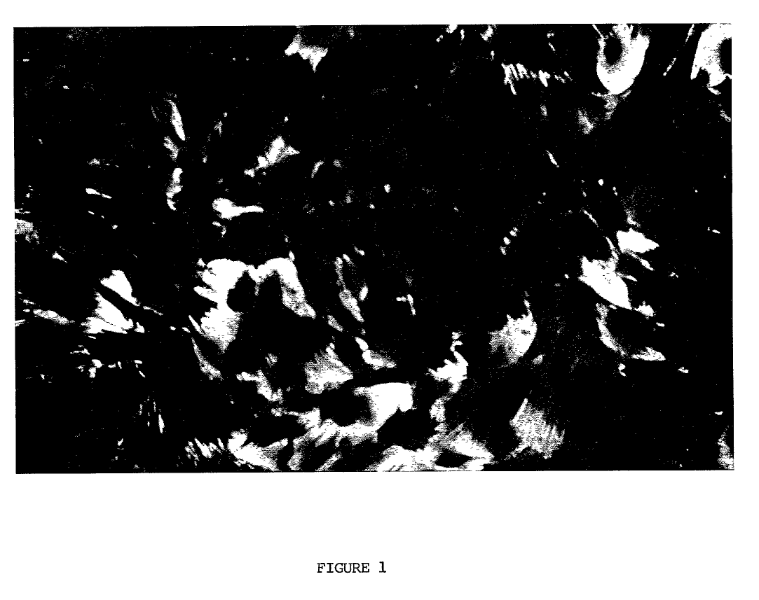

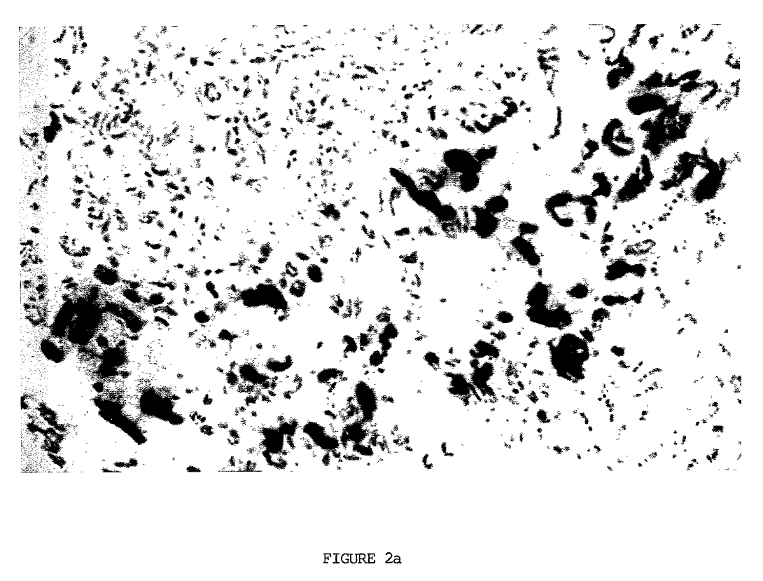

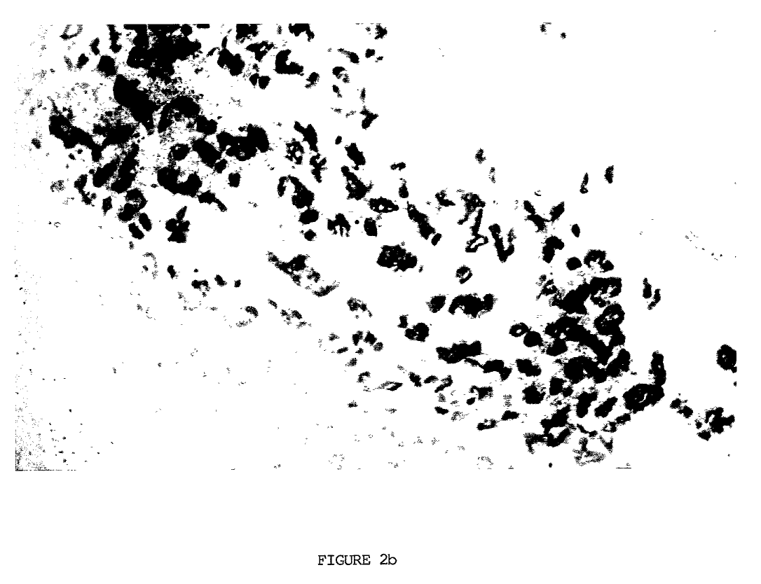

A significant X-gal staining was observed in more than 95% of the in vitro cultures at both the one week and three weeks. FIG. 1 shows the LacZ expression in in vitro cells three weeks after injection. The in vivo gene transfer group also demonstrated X-gal staining of nucleus pulposus cells at one week, three weeks and eight weeks. FIG. 2a shows the LacZ expression three weeks after in vivo injection and FIG. 2b shows LacZ expression eight weeks after injection. Positive X-gal staining was observed only in the nucleus pulposus of the injected lumber discs, not in the intervertebral discs of the adjacent levels. The control groups both in vitro and in vivo demonstrated negative X-gal staining.

This example demonstrates the feasibility and efficacy of adenovirus mediated gene transfer to the intervertebral disc. It was found that the expression of the marker gene introduced to intervertebral disc cells by adenovirus persisted at least 3 weeks both in vitro and in vivo.

### Example 2

Adenoviruses were used as vectors to facilitate cellular uptake of the genetic material in such a way that the genetic information would be expressed. Two different adenoviral constructs were used: (1) Ad/CMV-TGF-β1, encoding human TGF-β1, and (2) Ad/CMV-luciferase, encoding luciferase, an enzyme that catalyzes light production in bioluminescent organisms such as fireflies (this latter construct was used as a “control” adenovirus). The two adenoviral constructs used had the E1 and E3 regions of the adenovirus genome deleted and either the hTFG-β1 gene or luciferase gene was cloned into the E1 region of adenovirus under the control of the human CMV early promoter. The recombinant adenoviral constructs used in this study were grown in 293 cells and purified by CsCl density gradient purification.

Five skeletally mature female New Zealand white rabbits, about 4.0-5.0 lbs. each, were used in this study. The anterior aspects of the L2-3, L3-4, and L4-5 intervertebral discs were exposed using a retroperitoneal approach. In three rabbits (n=9 discs), 20 μl of saline containing Ad/CMV-hTGFβ1 (6×10⁶ PFU) were injected through a 26-gauge hypodermic needle into the nucleus pulposus. In the fourth and fifth rabbits (n=3 discs each), 20 μl of saline with or without Ad/CMV-luciferase (6×10⁶ PFU) were injected, respectively. For each rabbit, the L1-2 intervertebral disc was used as the intact control. After injection, all wounds were closed routinely. Postoperatively, the animals were allowed free cage activity (4000 sq. cm), food, and water. Butorphanol tartrate (50 mg/kg) IM and cephalothin sodium (9100-150 mg/kg) were administered twice per day for two days or until no longer needed. After one week, the animals were euthanized with an IM relaxant of ketamine (25.0 mg/kg) followed by an intravenous injection of sodium pentobarbital (1.2 g/kg). Upon sacrifice and harvest of the lumbar spines, all of the intervertebral discs appeared macroscopically normal.

After sacrifice, the nucleus pulposus tissues were harvested and cultured in Neuman-Tytell serumless medium (GIBCO, Grand Island, N.Y.) at 37° C. for 48 hours, after which the medium was extracted for ELISA (R&D Systems, Minneapolis, Minn.) to detect TGF-β1 production. New medium containing ³⁵S-sulfate (10 μCi/ml) was added to the cultures. Forty-eight hours later, the medium, together with nucleus pulposus tissue, was extracted and combined with an equal volume of solution containing 8M guanidine hydrochloride (GuHCl), 20 mM EDTA, and a mixture of proteinase inhibitors, and incubated at 60° C. for 48 hours. For quantitative evaluation of the ³⁵S-labeled proteoglycans, aliquots of the extracts were eluted on Sephadex G-25 in PD-10 columns (Pharmacia Biotech, Piscataway, N.J.) and after 24 hours incubation at room temperature, the radioactivity of the newly synthesized proteoglycans was measured by a scintillation counter (Packard #1900TR, Mariden, Conn.). All of the data were normalized by wet tissue weight, and statistical analysis performed using one-way ANOVA with Fisher's Protected LSD post-hoc test. Two discs from the Ad/CMV-hTGFβ1 group were excluded from the data analysis because of technical problems during surgery.

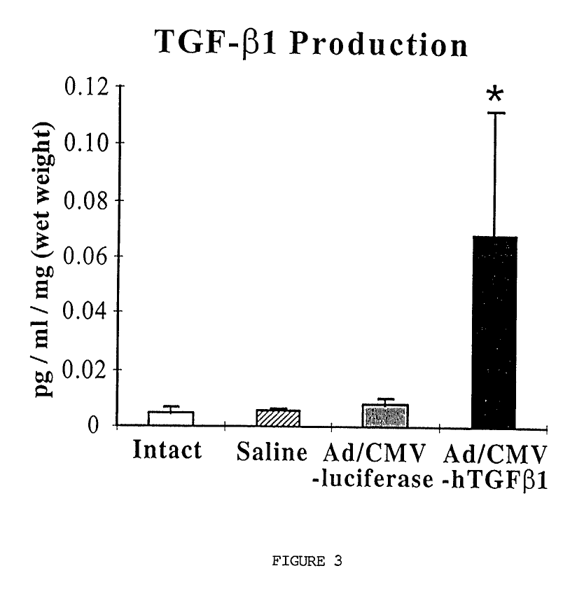

FIG. 3 shows the bioassay results from active TGF-β1 production in all treatment groups. The average TGF-β1 production in the intact discs as well as the discs that received a saline-only injection or Ad/CMV-luciferase injection was less than 0.01 pg/ml/mg wet weight. In contrast, average TGF-β1 production in the discs that received an AD/CMV-TGFβ1 injection was nearly 0.07 pg/ml/mg wet weight—significant increase over that of the control discs (p<0.05). It is important to note that this bioassay was for active TGF-β1, and that no artificial procedures (e.g., application of heat, acidity, etc.) were introduced to activate latent forms of TGF-β1.

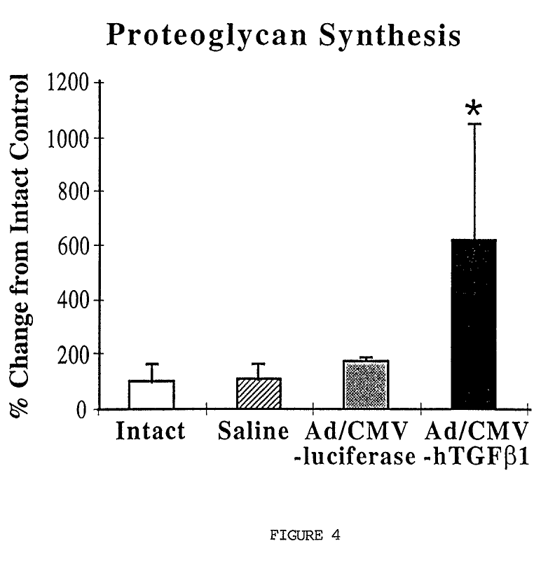

FIG. 4 shows the bioassay results for proteoglycan synthesis in all treatment groups. The average proteoglycan synthesis in the discs that received an Ad/CMV-TGFβ1 injection was significantly greater than that of the control discs (p<0.05). In fact, there was an over six-fold increase in proteoglycan synthesis for the Ad/CMV-TGFβ1 treatment group compared to the intact control group.

This example demonstrates that a therapeutic protein, specifically a growth factor, can be introduced to the intervertebral disc cells of a patient according to the present methods. Significantly, the disc cells will subsequently express biologically active gene products that provide a therapeutic benefit to the patient. Here, that benefit is the production of proteoglycan, which is known to contribute to the formation of matrix in disc cells.

What is claimed is:

1. A method for transferring a gene of interest to an intervertebral disc cell of a patient comprising:
introducing a nucleic acid sequence encoding a gene of interest to an intervertebral disc cell, such that said gene is expressed by said intervertebral disc cell.

2. The method of claim 1, wherein said method of introduction is selected from the group consisting of viral means and non-viral means.

3. The method of claim 2, wherein said viral means is a viral vector selected from the group consisting of a retroviral vector, adenoviral vector, adeno-associated viral vector, and herpes viral vector.

4. The method of claim 3, wherein said viral vector contains said nucleic acid sequence operatively linked to a promoter that is active in intervertebral disc cells.

5. The method of claim 2, wherein said non-viral means are selected from the group consisting of a liposome, calcium phosphate, electroporation, DEAE dextran and direct injection of naked nucleic acid.

6. The method of claim 1, wherein said nucleic acid sequence is selected from the group consisting of DNA, cDNA, RNA, oligonucleotides and an anti-sense nucleic acid molecule.

7. The method of claim 1, wherein said nucleic acid sequence is cDNA and said cDNA sequence is introduced to said intervertebral disc cell by an adenoviral vector; wherein said adenoviral vector contains said cDNA operatively linked to a promoter that is active in intervertebral disc cells.

8. The method of claim 7, wherein said promoter is a cytomegalovirus promoter.

9. A method for treating a degenerative disc disease in a patient comprising:
introducing an effective amount of a nucleic acid sequence encoding a gene of interest into a population of intervertebral disc cells, such that subsequent expression of said gene by at least one of said intervertebral disc cells in said patient reduces at least one pathology of degenerative disc disease in said patient.

10. The method of claim 9, wherein said method of introduction is selected from the group consisting of viral means and non-viral means.

11. The method of claim 10, wherein said viral means is a viral vector selected from the group consisting of a retroviral vector, adenoviral vector, adeno-associated viral vector, and herpes viral vector.

12. The method of claim 11, wherein said viral vector contains said nucleic acid sequence operatively linked to a promoter that is active in intervertebral disc cells.

13. The method of claim 9, wherein said non-viral means are selected from the group consisting of a liposome, calcium phosphate, electroporation, DEAE dextran and direct injection of naked nucleic acid.

14. The method of claim 9, wherein said nucleic acid sequence is selected from the group consisting of DNA, cDNA, RNA, oligonucleotides and an anti-sense nucleic acid molecule.

15. The method of claim 9, wherein said nucleic acid sequence is cDNA and said cDNA sequence is introduced to said intervertebral disc cell by an adenoviral vector; wherein said adenoviral vector contains said cDNA operatively linked to a promoter that is active in intervertebral disc cells.

16. The method of claim 15, wherein said promoter is a cytomegalovirus promoter.

17. The method of claim 9, wherein said gene of interest encodes a growth factor.

18. The method of claim 17, wherein said growth factor is selected from the group consisting of transforming growth factors, fibroblast growth factors, and insulin-like growth factors.

19. The method of claim 17, wherein said growth factor is bone morphogenetic protein.

20. The method of claim 9, wherein reduction of at least one pathology is effected by stimulating growth of disc matrix or disc matrix components.

21. The method of claim 9, wherein said gene of interest is selected from the group consisting of an interleukin-1 receptor antagonist protein, a soluble interleukin-1 receptor, a soluble tumor necrosis factor-α receptor, and a proteinase inhibitor.

22. The method of claim 21, wherein said proteinase inhibitor is selected from the group consisting of the tissue inhibitors of metalloproteinase, plasminogen activator inhibitors, and serpins.

23. The method of claim 9, wherein reduction of at least one pathology of degenerative disc disease disorder is effected by preventing further degradation of the disc.

24. The method of claim 18, wherein said growth factor is TGF-β1.

25. The method of claim 9, wherein said intervertebral disc cells are selected from annulus cells, nucleus pulposus cells or combinations thereof.

26. The method of claim 25, wherein said intervertebral disc cells are nucleus pulposus cells.

27. A method for producing an animal model for degenerative disc disease comprising:
introducing an effective amount of a nucleic acid sequence encoding a gene of interest into a population of intervertebral disc cells, such that subsequent expression of said gene by at least one of said intervertebral disc cells in said animal contributes to at least one pathology of degenerative disc disease in said animal.

28. The method of claim 27, wherein said method of introduction is selected from the group consisting of viral means and non-viral means.

29. The method of claim 28, wherein said viral means is a viral vector selected from the group consisting of a retroviral vector, adenoviral vector, adeno-associated viral vector, and herpes viral vector.

30. The method of claim 29, wherein said viral vector contains said nucleic acid sequence operatively linked to a promoter that is active in intervertebral disc cells.

31. The method of claim 28, wherein said non-viral means are selected from the group consisting of a liposome, calcium phosphate, electroporation, DEAE dextran and direct injection of naked nucleic acid sequences.

32. The method of claim 27, wherein said nucleic acid sequence is selected from the group consisting of DNA, cDNA, RNA, oligonucleotides and an anti-sense nucleic acid molecule.

33. The method of claim 27, wherein said nucleic acid sequence is cDNA and said cDNA sequence is introduced to said intervertebral disc cell by an adenoviral vector; wherein said adenoviral vector contains said cDNA operatively linked to a promoter that is active in intervertebral disc cells.

34. The method of claim 33, wherein said promoter is a cytomegalovirus promoter.

35. The method of claim 27, wherein said gene of interest is selected from the group consisting of interleukins, proteinases, tumor necrosis factors, and NO synthases.

36. The method of claim 35, wherein said proteinase is aggrecanase.

37. The method of claim 35, wherein said proteinase is a metalloproteinase selected from the group consisting of collagenase, gelatinase and stromelysin.

38. The method of claim 35, wherein said gene of interest is interleukin-1.

39. A genetically modified intervertebral disc cell which expresses an exogenous gene of interest.

40. The intervertebral disc cell of claim 39, wherein said cell is an annulus cell.

41. The intervertebral disc cell of claim 39, wherein said cell is a nucleus pulposus cell.

42. The intervertebral disc cell of claim 39, wherein said gene of interest encodes a protein that is useful in the treatment of a patient for a degenerative disc disorder.

43. The intervertebral disc cell of claim 39, wherein said gene of interest encodes a protein that causes a pathology associated with degenerative disc disorder.

44. The method of claim 9, wherein said gene of interest encodes growth hormone.

45. The method of claim 10, wherein said viral means or said non-viral means are directly injected into one or more discs of said patient.

46. The method of claim 10, wherein said viral means or said non-viral means are coated on or otherwise contained in a support device, and said support device is introduced to one or more discs of said patient.

47. The method of claim 46, wherein said support device is made from one or more members selected from the group consisting of collagen, glycosaminoglycans, polylactic acid polymers, calcium sulfate, tricalciumphosphate, hydroxyapatite, biphasic tricalciumphosphate/hydroxyapatite ceramic, polylactic acids, polylactate, polyanhydrides and chitosan.

48. The method of claim 28, wherein said viral means or said non-viral means are directly injected into one or more discs of said animal.

49. The method of claim 28, wherein said viral means or said non-viral means are coated on or otherwise contained in a support device, and said support device is introduced to one or more discs of said animal.

50. The method of claim 49, wherein said support device is made from one or more members selected from the group consisting of collagen, glycosaminoglycans, polylactic acid polymers, calcium sulfate, tricalciumphosphate, hydroxyapatite, biphasic tricalciumphosphate/hydroxyapatite ceramic, polylactic acids, polylactate, polyanhydrides and chitosan.
